(19) United States
(12) Patent Application Publication
(10) Pub. No.: US 2025/0099268 A1
(43) Pub. Date: Mar. 27, 2025

(54) PROSTHESIS AND ELECTRODE CONNECTOR FOR A PROSTHESIS

(71) Applicant: COAPT LLC, Chicago, IL (US)

(72) Inventor: Blair Andrew Lock, Wilmette, IL (US)

(21) Appl. No.: 18/832,544

(22) PCT Filed: Mar. 17, 2023
(86) PCT No.: PCT/US2023/015513

§ 371 Date: Jul. 24, 2024

Related U.S. Application Data
(60) Provisional application No. 63/321,967, filed on Mar. 21, 2022.

(51) Int. Cl. A61F 2/72; A61F 2/76

(52) U.S. Cl. CPC A61F 2/72; A61F 2/76

(57) ABSTRACT

An electrode connector includes an inner portion having a first head portion. The first head portion includes an arcuate outer surface and a generally planar inner surface, opposite the outer surface. At least one of an annular recess formed in the inner surface and/or an annular protrusion extends from the inner surface.

### FIELD OF THE INVENTION

This disclosure relates generally to prosthesis and, more particularly, to electrode connectors for prosthesis.

### BACKGROUND

Prostheses are air sealed to help in attachment of the prosthesis. However, installation of current electrode connectors through holes in the prosthesis can prevent or reduce creation of the desired air seal. Some attempts to seal groups of electrode connectors within a prosthesis have been attempted, however, these attempts require fairly large units to hold the electrode connectors and large and unwieldy sealing mechanisms in an attempt to create air tight seals. Therefore, there is a need for an improved electrode connector design for use in prostheses that self-seals and assists in securing the inner portion of the electrode to the prosthesis and does not require a user to trim and file down the end of the electrode during installation.

### SUMMARY

In accordance with one exemplary aspect of the present invention, an electrode connector comprises an inner portion comprising a first head portion. The first head portion comprises an arcuate outer surface and a generally planar inner surface, opposite the outer surface. At least one of an annular recess is formed in the inner surface and/or an annular protrusion extends from the inner surface

In further accordance with any one or more of the foregoing exemplary aspects of the present invention, an electrode connector may further include, in any combination, any one or more of the following preferred forms.

In one preferred form, first head portion includes the annular recess and the annular protrusion and the annular protrusion is positioned radially outward of the annular recess.

In another preferred form, the inner portion comprises a first body portion extending from the inner surface of the first head portion. The first body portion comprises a bore formed into the first body portion that has an internal surface that is at least partially threaded.

In another preferred form, the electrode connector comprises an outer portion comprising a second head portion and a second body portion. The second body portion extends from the second head portion and is configured to be received in the bore of the first body portion of the inner portion and has an external surface that is at least partially threaded and is configured to engage the at least partially threaded surface of the bore.

In another preferred form, the second head portion is configured to receive a tightening device.

In another preferred form, the electrode connector comprises an o-ring positioned in the annular recess of the first head portion.

In another preferred form, the first head portion has a generally circular perimeter and at least one planar surface formed on the perimeter.

In accordance with another exemplary aspect of the present invention, a prosthesis comprises a wall, including an inner surface and an outer surface, and an electrode connector. The electrode connector comprises an inner portion comprising a first head portion. The first head portion comprises an arcuate outer surface and a generally planar inner surface, opposite the outer surface, positioned adjacent an inner surface of a wall of the prosthesis. At least one of an annular recess is formed in the inner surface and/or an annular protrusion extends from the inner surface and engages the inner surface of the wall of the prosthesis to provide a seal between the first head portion of the inner portion and the inner surface of the wall of the prosthesis.

In further accordance with any one or more of the foregoing exemplary aspects of the present invention, a prosthesis may further include, in any combination, any one or more of the following preferred forms.

In one preferred form, the first head portion includes the annular recess and the annular protrusion and the annular protrusion is positioned radially outward of the annular recess.

In another preferred form, the inner portion comprises a first body portion extending from the inner surface of the first head portion. The first body portion comprises a bore formed into the first body portion that has an internal surface that is at least partially threaded.

In another preferred form, the prosthesis comprises an o-ring positioned in the annular recess of the first head portion that provides a seal between the head of the inner portion and the inner surface of the wall of the prosthesis.

In another preferred form, the prosthesis comprises an adhesive and/or sealant located within the annular recess of the first head portion that secures the inner portion of the electrode connector to the inner surface of the prosthesis and provides a seal between the first head portion of the inner portion and the inner surface of the wall of the prosthesis.

In another preferred form, the first head portion has a generally circular perimeter and at least one planar surface formed on the perimeter.

In another preferred form, the electrode connector comprises an outer portion positioned adjacent the outer surface of the wall of the prosthesis. The outer portion of the electrode connector comprises a second head portion and a second body portion extending from the second head portion. The second body portion has an external surface that is at least partially threaded and that is configured to engage the at least partially threaded surface of the bore.

In another preferred form, the second head portion is configured to receive a tightening device.

In another preferred form, the prosthesis comprises at least one of an o-ring, a washer, and/or a nut positioned between the second head portion of the outer portion of the electrode connector and the outer surface of the wall of the prosthesis that are configured to secure an electrode lead to the outer portion of the electrode connector.

In accordance with another exemplary aspect of the present invention, a method for sealing and securing an electrode connector to a prosthesis, comprising the steps of: positioning an inner surface of a first head portion of an inner portion of the electrode connector adjacent an inner surface of a wall of the prosthesis; and providing a seal between the first head portion of the inner portion of the electrode connector and the inner surface of the wall of the prosthesis via at least one of: an annular protrusion on the inner surface of the first head portion of the inner portion of the electrode connector engaging the inner surface of the wall of the prosthesis; and/or providing an adhesive or an o-ring in an annular recess in the inner surface of the first head portion of the inner portion of the electrode connector, the adhesive or o-ring engaging the inner portion of the electrode connector and the inner surface of the wall of the prosthesis.

In further accordance with any one or more of the foregoing exemplary aspects of the present invention, a method for sealing and securing an electrode connector to a prosthesis may further include, in any combination, any one or more of the following preferred forms.

In one preferred form, the method comprises the step of inserting a first body portion of an inner portion of the electrode connector through a hole formed in a wall of the prosthesis.

In another preferred form, the method comprises the step of threading a second body portion of an outer portion of the electrode connector into a threaded bore formed in the first body portion of the inner portion of the electrode connector.

In another preferred form, the method comprises the steps of: applying a rotational force to a second head portion of the outer portion of the electrode connector; and preventing rotational motion of the inner portion of the electrode connector.

In another preferred form, the second head portion of the outer portion of the electrode connector is configured to receive a tightening device.

In another preferred form, the method comprises the step of positioning an electrode lead between the second head portion of the outer portion of the electrode connector and the first body portion of the inner portion of the electrode connector.

In another preferred form, the method comprises the step of positioning at least one of an o-ring, a washer, and/or a nut between the second head portion of the outer portion of the electrode connector and an outer surface of the wall of the prosthesis to secure the electrode lead to the outer portion of the electrode connector.

### BRIEF DESCRIPTION OF THE DRAWINGS

The figures described below depict various aspects of the systems and devices disclosed therein. It should be understood that each figure depicts an embodiment of a particular aspect of the disclosed systems and devices, and that each of the figures is intended to accord with a possible embodiment thereof. Further, wherever possible, the following description refers to the reference numerals included in the following figures, in which features depicted in multiple figures are designated with consistent reference numerals.

There are shown in the drawings arrangements which are presently discussed, it being understood, however, that the present embodiments are not limited to the precise arrangements and instrumentalities shown, wherein:

FIG. 1 is a front view of an example prosthesis with multiple electrode connectors;

FIG. 2 is a cross-sectional view of the example prosthesis of FIG. 1 with a first example electrode connector, taken along line 2-2 in FIG. 1;

FIG. 3 is a perspective view of an inner portion of the electrode connector in FIG. 2;

FIG. 4 is a top view of the inner portion of FIG. 3;

FIG. 5 is a cross-sectional view of the example prosthesis of FIG. 1 with a second example electrode connector, taken along line 2-2 in FIG. 1;

FIG. 6 is a perspective view of an inner portion of the electrode connector in FIG. 5;

FIG. 7 is a top view of the inner portion of FIG. 6;

FIG. 8 is a cross-sectional view of the example prosthesis of FIG. 1 with a third example electrode connector, taken along line 2-2 in FIG. 1;

FIG. 9 is a perspective view of an inner portion of the electrode connector in FIG. 8; and

FIG. 10 is a top view of the inner portion of FIG. 9.

The figures depict preferred embodiments for purposes of illustration only.

Alternative embodiments of the systems and methods illustrated herein may be employed without departing from the principles of the invention described herein.

### DETAILED DESCRIPTION

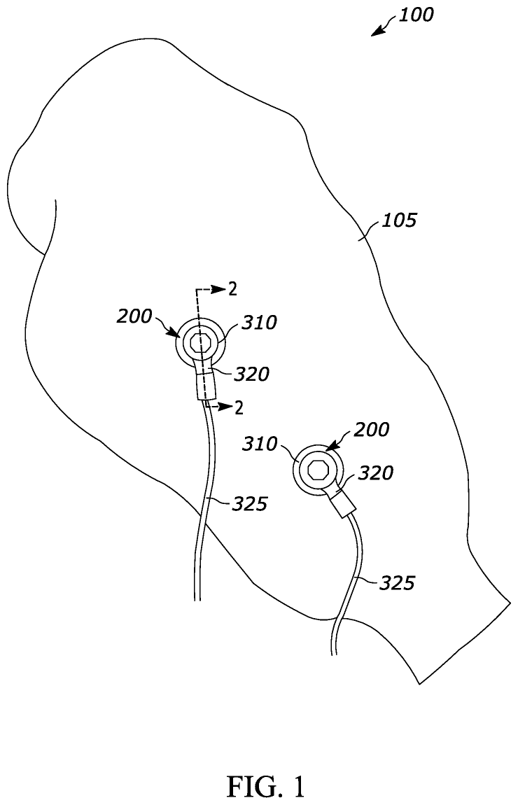

Referring to FIG. 1, a prosthesis 100 is shown that has a wall 105 and includes one or more electrode connectors 200. An inner surface 110 of wall 105 is configured to contact the skin of a user of prosthesis 100 and an outer surface 115 of wall is opposite inner surface 110. Electrode connectors 200 extend through corresponding holes 120 in wall 105 and are configured to receive biosignals from the body of the user. Each electrode connector 200 receives an electrode lead 320, which is connected to a controller (not shown) through a wire 325 to communicate the biosignals received by the electrode connector 200 to the controller. The controller may comprise one or more processors or memories which may comprise part of a system-on-a-chip (SoC), circuit board, or other integrated circuit. In various aspects, the electrode connectors detect or sense biosignals from the body of the user and transmit the biosignals to the one or more processors and/or memories. In this way, the electrode connectors 200 are communicatively coupled to the controller for the transmission of biosignal data thereto. The biosignal may be used for control of the prosthesis 100 by the controller or for other purposes, including, by way of non-limiting example, data collection or profiling of the user manipulating the prosthesis device 100.

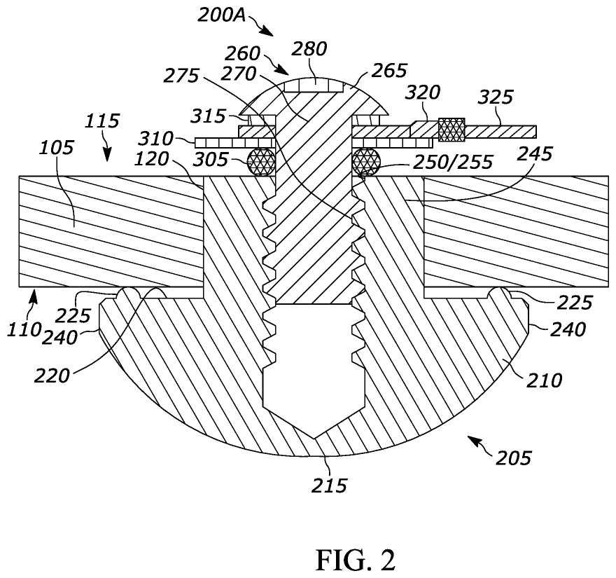

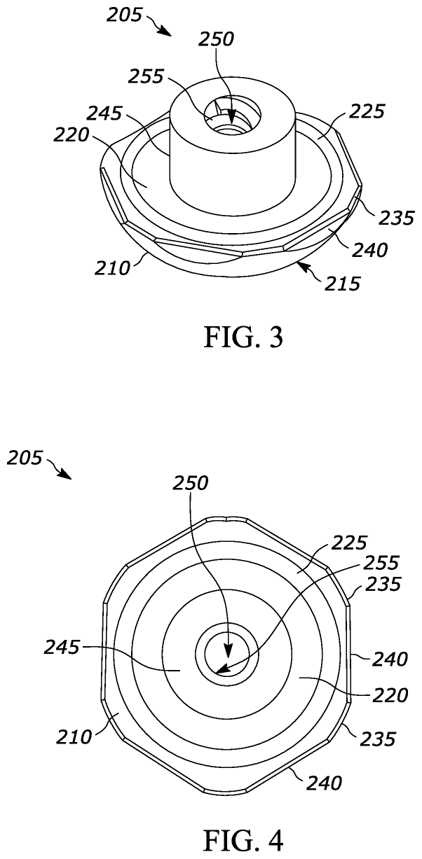

Referring to FIGS. 2-4, a first example electrode connector 200A is shown that can be used in prosthesis 100. Electrode connector 200A includes an inner portion 205 that is configured to contact and receive the biosignals from the body of the user. Inner portion 205 has a first head portion 210 that has a smooth arcuate outer surface 215 to contact the body of the user and a generally planar inner surface 220 that is positioned adjacent inner surface 110 of wall 105 of prosthesis 100. First head portion 210 can have a generally circular perimeter 235 and, if desired, one or more planar surfaces 240 can be formed in perimeter 235 and configured to receive a tool to assist in holding inner portion 205 of electrode connector 200A during installation. In addition, to provide a seal between first head portion 210 of inner portion 205 of electrode connector 200A and inner surface 110 of wall 105 of prosthesis 100 and prevent air from entering or escaping through hole 120 and possibly breaking a vacuum formed within prosthesis 100 when worn by the user, first head portion 210 has an annular protrusion 225 that extends from inner surface 220, which engages inner surface 110 of wall 105 to provide the seal.

In the particular embodiment shown, first head portion 210 of electrode connector 200A also includes a first body portion 245 that extends from inner surface 220 of first head portion 210. First body portion 245 extends through hole 120 in wall 105 and has a bore 250 that has an internal surface 255 that is at least partially threaded.

To assist in securing inner portion 205 to wall 105 of prosthesis 100 and to connect inner portion 205 to electrode lead 320, electrode connector 200A can also include an outer portion 260 that is positioned adjacent outer surface 115 of wall 105 and provides an electrically conductive connection between inner portion 205 and electrode lead 320. In the example shown, outer portion 260 includes a second head portion 265 and a second body portion 270 extending from second head portion 265. Second body portion 270 includes an external surface 275 that is at least partially threaded and is configured to engage the at least partially threaded internal surface 255 of bore 250 to connect and secure outer portion 260 to inner portion 205. Second head portion 265 of outer portion 260 can also be configured to receive a tightening device or tool, such as an allen wrench, a screw driver, a socket wrench, etc. to assist in applying rotational force to second head portion 265 to tighten outer portion 260 during assembly. For example, second head portion 265 can include a polygonal cavity 280 that is configured to receive an allen wrench.

To assist in making the electrically conducive connection between inner portion 205 and electrode lead 320 and to assist in securing electrode lead 320 to outer portion 260, at least one of an o-ring 305, a washer 310, and/or a nut 315 can be positioned between second head portion 265 of outer portion 260 and outer surface 115 of wall 105 of prosthesis 100. If desired, in addition to providing an air tight seal between inner portion 205 of electrode connector 200A, an air tight seal can also be formed between second head portion 265 of outer portion 260 and outer surface 115 of wall 105 of prosthesis 100 as well.

In use, to seal and secure electrode connector 200A to wall 105 of prosthesis 100, inner surface 220 of first head portion 210 of inner portion 205 is positioned adjacent, and preferably in contact with, inner surface 110 of wall 105 of prosthesis 100. The seal between first head portion 210 of inner portion 205 and inner surface 110 of wall 105 is provided by annular protrusion 225 engaging inner surface 110 of wall 105.

If applicable, first body portion 245 of inner portion 205 can also be inserted through hole 120 in wall 105. In the example shown, at least one of o-ring 305, washer 310, and/or nut 315 can be positioned on outer portion 260 along with electrode lead 320 to secure electrode lead 320 to outer portion 260 and second body portion 270 of outer portion 260 can be threaded into bore 250 of first body portion 245 of inner portion 205. During this portion of the installation, inner portion 205 can be held in place to prevent rotational motion and a rotational force can be applied to second head portion 265 of outer portion 260, for example by a tightening device as describe above, to tighten the connection between inner portion 205 and outer portion 260. In addition, as discussed above, in addition to provide an air tight seal between inner portion 205 and inner surface 110 of wall 105 an air tight seal can also be formed between second head portion 265 of outer portion 260 and outer surface 115 of wall 105.

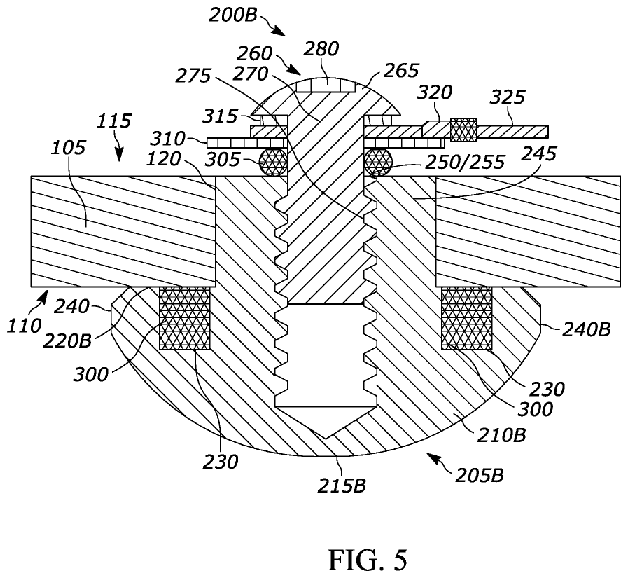

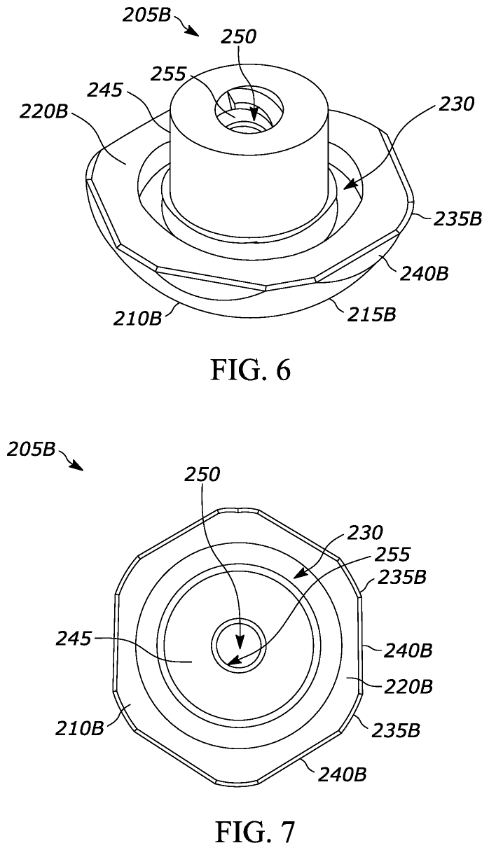

Referring to FIGS. 5-7, a second example electrode connector 200B is shown that can be used in prosthesis 100. Electrode connector 200B includes an inner portion 205B that is configured to contact and receive the biosignals from the body of the user. Inner portion 205B has a first head portion 210B that has a smooth arcuate outer surface 215B to contact the body of the user and a generally planar inner surface 220B that is positioned adjacent inner surface 110 of wall 105 of prosthesis 100. First head portion 210B can have a generally circular perimeter 235B and, if desired, one or more planar surfaces 240B can be formed in perimeter 235B and configured to receive a tool to assist in holding inner portion 205B of electrode connector 200B during installation. In addition, to provide a seal between first head portion 210B of inner portion 205B of electrode connector 200B and inner surface 110 of wall 105 of prosthesis 100 and prevent air from entering or escaping through hole 120 and possibly breaking a vacuum formed within prosthesis 100 when worn by the user, first head portion 210 has an annular recess 230 formed in inner surface 220B. An adhesive and/or sealant 300 can be located with annular recess 230 to assist in securing inner portion 205B to inner surface 110 of wall 105 and to provide the seal between first head portion 210B and inner surface 110 of wall 105. Alternatively, rather than adhesive and/or sealant 300, an o-ring can be positioned in annular recess 230 to provide the seal between first head portion 210B and inner surface 110 of wall 105.

In the particular embodiment shown, first head portion 210B of electrode connector 200B also includes first body portion 245 that extends from inner surface 220B of first head portion 210B. First body portion 245 extends through hole 120 in wall 105 and has a bore 250 that has an internal surface 255 that is at least partially threaded.

To assist in securing inner portion 205B to wall 105 of prosthesis 100 and to connect inner portion 205B to electrode lead 320, electrode connector 200B can also include an outer portion 260 that is positioned adjacent outer surface 115 of wall 105 and provides an electrically conductive connection between inner portion 205B and electrode lead 320. In the example shown, outer portion 260 includes a second head portion 265 and a second body portion 270 extending from second head portion 265. Second body portion 270 includes an external surface 275 that is at least partially threaded and is configured to engage the at least partially threaded internal surface 255 of bore 250 to connect and secure outer portion 260 to inner portion 205B. Second head portion 265 of outer portion 260 can also be configured to receive a tightening device or tool, such as an allen wrench, a screw driver, a socket wrench, etc. to assist in applying rotational force to second head portion 265 to tighten outer portion 260 during assembly. For example, second head portion 265 can include a polygonal cavity 280 that is configured to receive an allen wrench.

To assist in making the electrically conducive connection between inner portion 205B and electrode lead 320 and to assist in securing electrode lead 320 to outer portion 260, at least one of o-ring 305, washer 310, and/or nut 315 can be positioned between second head portion 265 of outer portion 260 and outer surface 115 of wall 105 of prosthesis 100. If desired, in addition to providing an air tight seal between inner portion 205B of electrode connector 200B, an air tight seal can also be formed between second head portion 265 of outer portion 260 and outer surface 115 of wall 105 of prosthesis 100 as well.

In use, to seal and secure electrode connector 200B to wall 105 of prosthesis 100, adhesive and/or sealant 300, or alternatively an o-ring, is positioned in annular recess 230 and inner surface 220B of first head portion 210B of inner portion 205B is positioned adjacent, and preferably in contact with, inner surface 110 of wall 105 of prosthesis 100, such that adhesive and/or sealant 300, or the o-ring, engage inner portion 205B of electrode connector 200B and inner surface 110 of wall 105 of prosthesis 100. The seal between first head portion 210B of inner portion 205B and inner surface 110 of wall 105 is provided by adhesive and/or sealant 300, or the o-ring, engaging inner surface 110 of wall 105.

If applicable, first body portion 245 of inner portion 205B can also be inserted through hole 120 in wall 105. In the example shown, at least one of o-ring 305, washer 310, and/or nut 315 can be positioned on outer portion 260 along with electrode lead 320 to secure electrode lead 320 to outer portion 260 and second body portion 270 of outer portion 260 can be threaded into bore 250 of first body portion 245 of inner portion 205B. During this portion of the installation, inner portion 205B can be held in place to prevent rotational motion and a rotational force can be applied to second head portion 265 of outer portion 260, for example by a tightening device as describe above, to tighten the connection between inner portion 205B and outer portion 260. In addition, as discussed above, in addition to provide an air tight seal between inner portion 205B and inner surface 110 of wall 105 an air tight seal can also be formed between second head portion 265 of outer portion 260 and outer surface 115 of wall 105.

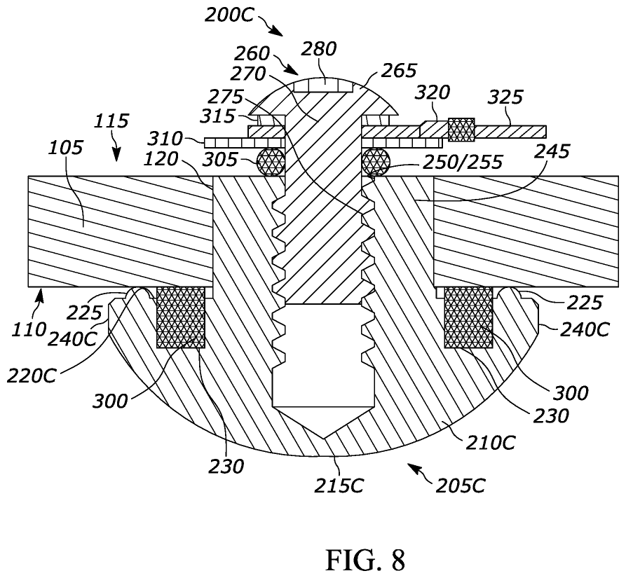

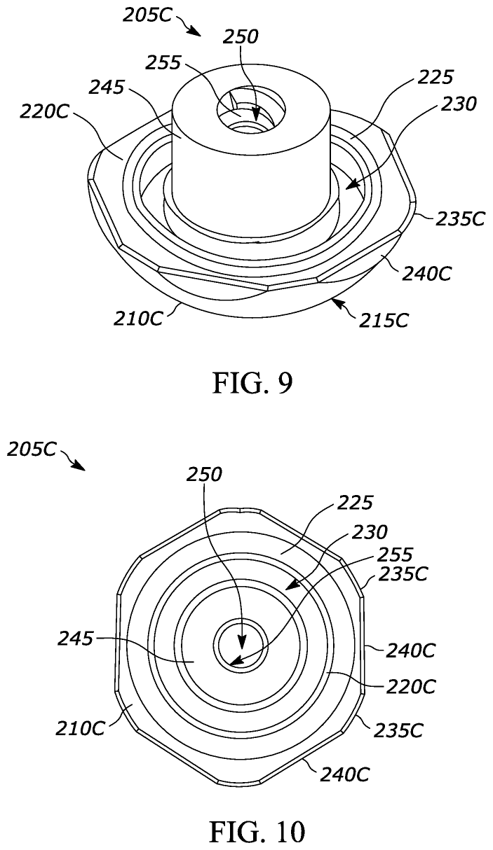

Referring to FIGS. 8-10, a third example electrode connector 200C is shown that can be used in prosthesis 100. Electrode connector 200C includes an inner portion 205C that is configured to contact and receive the biosignals from the body of the user. Inner portion 205C has a first head portion 210C that has a smooth arcuate outer surface 215C to contact the body of the user and a generally planar inner surface 220C that is positioned adjacent inner surface 110 of wall 105 of prosthesis 100. First head portion 210C can have a generally circular perimeter 235C and, if desired, one or more planar surfaces 240C can be formed in perimeter 235C and configured to receive a tool to assist in holding inner portion 205C of electrode connector 200C during installation. In addition, to provide a seal between first head portion 210C of inner portion 205C of electrode connector 200C and inner surface 110 of wall 105 of prosthesis 100 and prevent air from entering or escaping through hole 120 and possibly breaking a vacuum formed within prosthesis 100 when worn by the user, first head portion 210C has annular protrusion 225 extending from inner surface 220C, which engages inner surface 110 of wall 105 to provide the seal, and annular recess 230 formed in inner surface 220C. Preferably, annular protrusion 225 is positioned radially outward of annular recess 230, as shown. However, annular protrusion 225 can also be positioned radially inward of annular recess 230, if desired. An adhesive and/or sealant 300 can be located with annular recess 230 to assist in securing inner portion 205C to inner surface 110 of wall 105 and to provide the seal between first head portion 210C and inner surface 110 of wall 105. Alternatively, rather than adhesive and/or sealant 300, an o-ring can be positioned in annular recess 230 to provide the seal between first head portion 210C and inner surface 110 of wall 105 . . .

In the particular embodiment shown, first head portion 210C of electrode connector 200C also includes first body portion 245 extending from inner surface 220C of first head portion 210C. First body portion 245 extends through hole 120 in wall 105 and has a bore 250 that has an internal surface 255 that is at least partially threaded.

To assist in securing inner portion 205C to wall 105 of prosthesis 100 and to connect inner portion 205C to electrode lead 320, electrode connector 200C can also include an outer portion 260 that is positioned adjacent outer surface 115 of wall 105 and provides an electrically conductive connection between inner portion 205C and electrode lead 320. In the example shown, outer portion 260 includes a second head portion 265 and a second body portion 270 extending from second head portion 265. Second body portion 270 includes an external surface 275 that is at least partially threaded and is configured to engage the at least partially threaded internal surface 255 of bore 250 to connect and secure outer portion 260 to inner portion 205C. Second head portion 265 of outer portion 260 can also be configured to receive a tightening device or tool, such as an allen wrench, a screw driver, a socket wrench, etc. to assist in applying rotational force to second head portion 265 to tighten outer portion 260 during assembly. For example, second head portion 265 can include a polygonal cavity 280 that is configured to receive an allen wrench.

To assist in making the electrically conducive connection between inner portion 205C and electrode lead 320 and to assist in securing electrode lead 320 to outer portion 260, at least one of o-ring 305, washer 310, and/or a nut 315 can be positioned between second head portion 265 of outer portion 260 and outer surface 115 of wall 105 of prosthesis 100. If desired, in addition to providing an air tight seal between inner portion 205C of electrode connector 200C, and air tight seal can also be formed between second head portion 265 of outer portion 260 and outer surface 115 of wall 105 of prosthesis 100 as well.

In use, to seal and secure electrode connector 200C to prosthesis, adhesive and/or sealant 300, or alternatively an o-ring, is positioned in annular recess 230 and inner surface 220C of first head portion 210C of inner portion 205C is positioned adjacent, and preferably in contact with, inner surface 110 of wall 105 of prosthesis 100, such that adhesive and/or sealant 300, or the o-ring, engage inner portion 205C of electrode connector 200C and inner surface 110 of wall 105 of prosthesis 100 and annular protrusion 225 engages inner portion 205C of electrode connector 200C. The seal between first head portion 210C of inner portion 205C and inner surface 110 of wall 105 can be provided by annular protrusion 225 engaging inner surface 110 of wall 105 and by adhesive and/or sealant 300, or the o-ring, engaging inner surface 110 of wall 105.

If applicable, first body portion 245 of inner portion 205C can also be inserted through hole 120 in wall 105. In the example shown, at least one of o-ring 305, washer 310, and/or nut 315 can be positioned on outer portion 260 along with electrode lead 320 to secure electrode lead 320 to outer portion 260 and second body portion 270 of outer portion 260 can be threaded into bore 250 of first body portion 245 of inner portion 205C. During this portion of the installation, inner portion 205C can be held in place to prevent rotational motion and a rotational force can be applied to second head portion 265 of outer portion 260, for example by a tightening device as describe above, to tighten the connection between inner portion 205C and outer portion 260. In addition, as discussed above, in addition to provide an air tight seal between inner portion 205C and inner surface 110 of wall 105 an air tight seal can also be formed between second head portion 265 of outer portion 260 and outer surface 115 of wall 105.

While various embodiments have been described above, this disclosure is not intended to be limited thereto. Variations can be made to the disclosed embodiments that are still within the scope of the appended claims.

The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above described embodiments without departing from the scope of the invention, and that such modifications, alterations, and combinations are to be viewed as being within the ambit of the inventive concept.

The patent claims at the end of this patent application are not intended to be construed under 35 U.S.C. § 112 (f) unless traditional means-plus-function language is expressly recited, such as “means for” or “step for” language being explicitly recited in the claim(s).

What is claimed is:

1. An electrode connector, comprising:
an inner portion comprising a first head portion, the first head portion comprising:
an arcuate outer surface and a generally planar inner surface, opposite the outer surface; and
at least one of an annular recess formed in the inner surface and/or an annular protrusion extending from the inner surface.

2. The electrode connector of claim 1, wherein the first head portion includes the annular recess and the annular protrusion and the annular protrusion is positioned radially outward of the annular recess.

3. The electrode connector of claim 1, wherein the inner portion comprises a first body portion extending from the inner surface of the first head portion, the first body portion comprising a bore formed into the first body portion, the bore having an internal surface that is at least partially threaded.

4. The electrode connector of claim 3, comprising an outer portion, the outer portion comprising:
a second head portion; and
a second body portion extending from the second head portion, the second body portion configured to be received in the bore of the first body portion of the inner portion and having an external surface that is at least partially threaded and is configured to engage the at least partially threaded surface of the bore.

5. The electrode connector of claim 4, wherein the second head portion is configured to receive a tightening device.

6. The electrode connector of claim 1, comprising an o-ring positioned in the annular recess of the first head portion.

7. The electrode connector of claim 1, wherein the first head portion has a generally circular perimeter and at least one planar surface formed on the perimeter.

8. A prosthesis, comprising:
a wall including an inner surface and an outer surface; and
an electrode connector, the electrode connector comprising:
an inner portion comprising a first head portion, the first head portion comprising:
an arcuate outer surface and a generally planar inner surface, opposite the outer surface, the inner surface positioned adjacent the inner surface of the wall of the prosthesis; and
at least one of an annular recess formed in the inner surface and/or an annular protrusion extending from the inner surface and engaging the inner surface of the wall of the prosthesis to provide a seal between the first head portion of the inner portion and the inner surface of the wall of the prosthesis.

9. The prosthesis of claim 8, wherein the first head portion includes the annular recess and the annular protrusion and the annular protrusion is positioned radially outward of the annular recess.

10. The prosthesis of claim 8, wherein the inner portion comprises a first body portion extending from the inner surface of the first head portion, the first body portion comprising a bore formed into the first body portion, the bore having an internal surface that is at least partially threaded.

11. The prosthesis of claim 8, comprising an o-ring positioned in the annular recess of the first head portion and providing a seal between the first head portion of the inner portion and the inner surface of the wall of the prosthesis.

12. The prosthesis of claim 8, comprising an adhesive and/or sealant located within the annular recess of the first head portion, the adhesive and/or sealant securing the inner portion of the electrode connector to the inner surface of the prosthesis and providing a seal between the first head portion of the inner portion and the inner surface of the wall of the prosthesis.

13. The prosthesis of claim 8, wherein the first head portion has a generally circular perimeter and at least one planar surface formed on the perimeter.

14. The prosthesis of claim 10, wherein the electrode connector comprises an outer portion positioned adjacent the outer surface of the wall of the prosthesis, the outer portion of the electrode connector comprising:
a second head portion; and
a second body portion extending from the second head portion, the second body portion having an external surface that is at least partially threaded and that is configured to engage the at least partially threaded surface of the bore.

15. The prosthesis of claim 14, wherein the second head portion is configured to receive a tightening device.

16. The prosthesis of claim 14, comprising at least one of an o-ring, a washer, and/or a nut positioned between the second head portion of the outer portion of the electrode connector and the outer surface of the wall of the prosthesis, the o-ring, the washer, and/or the nut are configured to secure an electrode lead to the outer portion of the electrode connector.

17. A method for sealing and securing an electrode connector to a prosthesis, comprising the steps of:
positioning an inner surface of a first head portion of an inner portion of the electrode connector adjacent an inner surface of a wall of the prosthesis; and
providing a seal between the first head portion of the inner portion of the electrode connector and the inner surface of the wall of the prosthesis via at least one of:
an annular protrusion on the inner surface of the first head portion of the inner portion of the electrode connector engaging the inner surface of the wall of the prosthesis; and/or
providing an adhesive or an o-ring in an annular recess in the inner surface of the first head portion of the inner portion of the electrode connector, the adhesive or o-ring engaging the inner portion of the electrode connector and the inner surface of the wall of the prosthesis.

18. The method of claim 17, comprising the step of inserting a first body portion of the inner portion of the electrode connector through a hole formed in a wall of the prosthesis.

19. The method of claim 18, comprising the step of threading a second body portion of an outer portion of the electrode connector into a threaded bore formed in the first body portion of the inner portion of the electrode connector.

20. The method of claim 19, comprising the steps of:
applying a rotational force to a second head portion of the outer portion of the electrode connector; and
preventing rotational motion of the inner portion of the electrode connector.

21. The method of claim 20, wherein the second head portion of the outer portion of the electrode connector is configured to receive a tightening device.

22. The method of claim 20, comprising the step of positioning an electrode lead between the second head portion of the outer portion of the electrode connector and the first body portion of the inner portion of the electrode connector.

23. The method of claim 22, comprising the step of positioning at least one of an o-ring, a washer, and/or a nut between the second head portion of the outer portion of the electrode connector and an outer surface of the wall of the prosthesis to secure the electrode lead to the outer portion of the electrode connector.
